(19) United States
(12) Patent Application Publication
(10) Pub. No.: US 2019/0105112 A1
(43) Pub. Date: Apr. 11, 2019

(54) IMAGE GUIDED ROBOT FOR CATHETER PLACEMENT

(71) Applicant: KONINKLIJKE PHILIPS N.V., EINDHOVEN (NL)

(72) Inventors: Aleksandra POPOVIC, BOSTON, MA (US); David Paul NOONAN, NEW YORK, NY (US)

(21) Appl. No.: 16/086,805

(22) PCT Filed: Mar. 28, 2017
(86) PCT No.: PCT/EP2017/057316

§ 371 Date: Sep. 20, 2018

Related U.S. Application Data
(60) Provisional application No. 62/315,785, filed on Mar. 31, 2016.

(51) Int. Cl. A61B 34/30

(52) U.S. Cl. CPC A61B 34/30; A61B 2034/2065

(57) ABSTRACT

A robot includes a steerable device (402) having one or more robotically controlled joints configured to steer the steerable device. A device control system (430) is configured to adjust positioning of the steerable device in accordance with one of image feedback from an image control system (406) or a plan in a volume such that control commands are issued to the one or more robotically controlled joints to steer the steerable device in a direction consistent with navigation of the steerable device toward a target.

### BACKGROUND

### Technical Field

This disclosure relates to medical instruments, and more particularly to systems and methods for robotically steering a device using controlled joints in medical applications.

### Description of the Related Art

Balloon sinuplasty is a procedure in which a balloon catheter is inserted into a blocked sinus to relieve patients from symptoms of a sinus infection. During this procedure, a guide catheter is inserted through the nose into the sinus. The guide catheter can have curved tips to facilitate entry into an appropriate sinus. A guidewire is placed inside the catheter, and the guide catheter is retracted once the guidewire is in the right place. A balloon catheter is placed over the guidewire, and a balloon is inflated to open up air passageways. This procedure is done under the guidance of a flexible endoscope and X-rays. The X-rays are typically employed to verify that the guidewire is placed into an appropriate sinus opening.

The anatomy of sinuses is very complex and can include multiple sharp turns to reach a sinus cavity from the nose. In addition, finding an appropriate location for deploying the balloon is needed for the success of the therapy. The navigation is further hindered by some of the following described issues. For example, control of the guide catheter is complex. A surgeon needs to choose an appropriate angle for the curved tip, which is determined from a patient's computed tomography (CT) scan. The guide catheter is then pivoted and rotated to position the curve at the sinus entry point. The procedure is performed under image guidance, which may include a fiber optic endoscope inserted through the guide catheter and/or a C-arm X-ray system taking two dimensional images of the anatomy and the device. The X-ray guidance can be challenging since the 2D images cannot capture complex 3D anatomy. The endoscope guidance can show the sinus opening only if it is in front of the catheter.

### SUMMARY

In accordance with the present principles, a robot includes a steerable device having one or more robotically controlled joints configured to steer the steerable device. A device control system is configured to adjust positioning of the steerable device in accordance with one of image feedback from an image control system or a plan in a volume such that control commands are issued to the one or more robotically controlled joints to steer the steerable device in a direction consistent with navigation of the steerable device toward a target.

A guidance system includes a steerable device having an adjustable tip portion, the tip portion being coupled to a robotically controlled joint. An image control system is configured to combine intraoperative images with preoperative images to evaluate a position of the steerable device within a volume. A device control system is configured to receive position information from the image control system and to evaluate positioning of the steerable device in the volume using a kinematic model. The device control system issues control commands to the robotically controlled joint to steer the steerable device in a direction consistent with navigation of the steerable device toward a target.

A guidance method includes inserting a steerable device having an adjustable robotically controlled joint configured to be steered into a volume; providing position or image feedback of the steerable device within the volume; and automatically navigating the steerable device toward a target in accordance with a plan using a device control system configured to receive the feedback, to evaluate positioning of the steerable device in the volume and to issue control commands to the robotically controlled joint to steer the steerable device.

These and other objects, features and advantages of the present disclosure will become apparent from the following detailed description of illustrative embodiments thereof, which is to be read in connection with the accompanying drawings.

### BRIEF DESCRIPTION OF DRAWINGS

This disclosure will present in detail the following description of preferred embodiments with reference to the following figures wherein:

FIG. 1 is a block/flow diagram showing a guidance system which employs a steerable device having a robotically controlled joint to form a steerable tip portion on a medical device in accordance with one embodiment;

FIG. 2 is a flow diagram showing methods for guiding a steerable device (e.g., robot controlled) in accordance with illustrative embodiments

FIG. 3 is a diagram showing an illustrative joint with three degrees of rotational freedom and translation in accordance with one embodiment;

FIG. 4A is a diagram showing a steerable device approaching a branching structure in accordance with one embodiment;

FIG. 4B is a diagram showing the steerable device of FIG. 4A after being adjusted to select a desired pathway in accordance with the one embodiment; and

FIG. 5 is a block/flow diagram showing a robot which employs a steerable device and a device control system in accordance with another embodiment.

### DETAILED DESCRIPTION OF EMBODIMENTS

In accordance with the present principles, systems and methods are provided for a steerable device, which may include an actuated robotically controlled joint that is guided using an image guidance system to place a guidewire in a sinus or other complex cavity or lumen network. The steerable device may include one or more joints and may be referred to as a robot. The joints are configured to alter the shape of the steerable device to guide the device into a correct passageway. A guidewire can be placed through the lumen of the steerable device. The image control system performs integration of preoperative and intraoperative images and determines, from the images, the location in an anatomy where a steerable tip has to be guided and an angle of steering.

It should be understood that the present invention will be described in terms of medical instruments; however, the teachings of the present invention are much broader and are applicable to any steerable instruments for use in any portions of the body. In some embodiments, the present principles are employed in tracking or analyzing complex biological or mechanical systems. In particular, the present principles are applicable to internal tracking and operating procedures of biological systems and procedures in all areas of the body such as the lungs, brain, heart, gastro-intestinal tract, excretory organs, blood vessels, etc. The elements depicted in the FIGS. may be implemented in various combinations of hardware and software and provide functions which may be combined in a single element or multiple elements.

The functions of the various elements shown in the FIGS. can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W), Blu-Ray™ and DVD.

Reference in the specification to “one embodiment” or “an embodiment” of the present principles, as well as other variations thereof, means that a particular feature, structure, characteristic, and so forth described in connection with the embodiment is included in at least one embodiment of the present principles. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

It will also be understood that when an element such as an element, region or material is referred to as being “on” or “over” another element, it can be directly on the other element or intervening elements may also be present. In contrast, when an element is referred to as being “directly on” or “directly over” another element, there are no intervening elements present. It will also be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present.

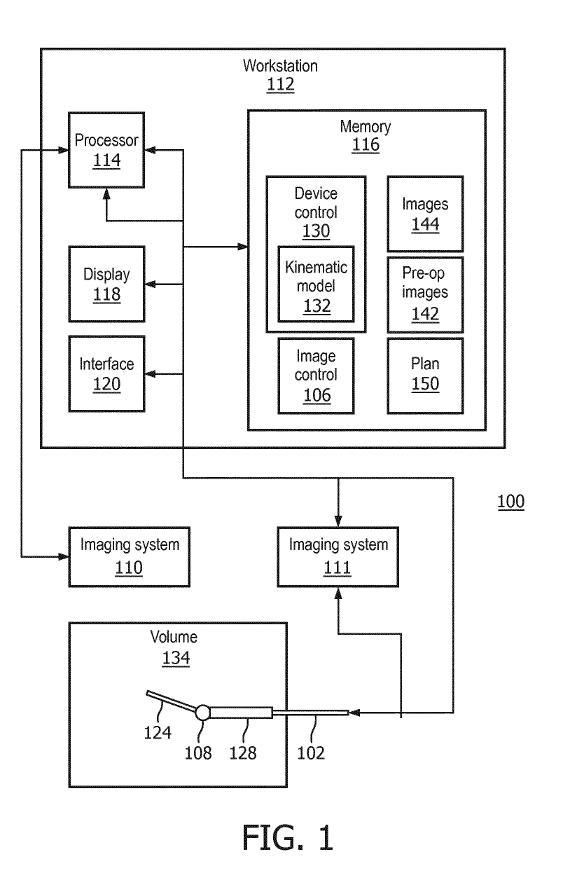

Referring now to the drawings in which like numerals represent the same or similar elements and initially to FIG. 1, a system 100 for robotic guidance in tissue in a subject is illustratively shown in accordance with one embodiment. System 100 may include a workstation or console 112 from which a procedure is supervised and/or managed. Workstation 112 preferably includes one or more processors 114 and memory 116 for storing programs and applications. Memory 116 may store a device control system 130 configured to control movement and programming of an actuated robot joint or joints 108 and other possible robotically controlled features in accordance with user input and/or feedback provided from one or more inputs. The system 100 includes a steerable device 102 and an image guidance or control system 106 to permit placement of a guidewire in a complex or branching network of tubes or cavities, e.g., sinus cavities, etc. The actuated device 102 may include one or more joints 108. The joints 108 are configured to steer a tip of the steerable device 102. The image control system or image guidance system 106 performs integration of preoperative images 142 and intraoperative images 144 and determines, from the images (142, 144), the location in anatomy where a steerable tip 124 of the device 102 (e.g., a catheter or catheter-like device) has to be steered and an angle of steering.

In one embodiment, the steerable device 102 may be fixed in space at a proximal end (for example using a medical positioning arm). A coordinate frame for each joint 108 can be defined in a coordinate system at the proximal end (fixed coordinate system). Since a position of each motor (not shown) for each joint 108 is known from motor encoders, position and three angles of orientation of each joint 108 is known in the fixed coordinate system as well.

To register this view with 3D position of the device 102 in the fixed coordinate frame, correspondence between joint positions in the fixed coordinate system and in X-ray images (or other images) is established. In the image taken by an imaging system 111, each rigid segment can be detected using image processing methods known in art, such as thresholding segmentation and shape fitting. Alternatively, a radiopaque marker can be attached to each joint 108.

In one embodiment, after the joints 108 are detected, they may be ordered in a simple tree where a parent and a child of a node are direct neighbors of any given joint. Given the linked architecture of the device 102, there will be two possible trees (proximal to distal and distal to proximal). Then, two correspondences are defined according to two trees. If a number of nodes in the tree is the same as the number of joints 108 of the device 102, two registrations need to be computed. If number of visible nodes (m) is smaller than total number of nodes (n), the number of possible registrations will be 2x(n choose m).

The registration process assumes m points in 2D X-ray space and m points in 3D robot space (fixed coordinate system). The registration process also assumes that focal length or the X-ray system is known. The pose of an X-ray detector of system 111 in the coordinate frame of the device 102 can thus be detected using any method known in art, such as iterative closest point, RANSAC (Random sample consensus) based iterative method, etc.

If m<n, residual error reported for correspondences that are not correct will be significantly higher than residual error from correct correspondences and those can be rejected using residual error as criteria. For example, a solution with the best residual error can be shown to the user as the position of X-ray system 111 with respect to the device 102. In case of flipped joint order, the user can select the right solution by observing rendering of both solutions or answering a simple question (e.g., “Is image detector above or below the patient?”). Other registration methods may also be employed to register intraoperative images 144 and preoperative images 142 and the steerable device 102.

The system 100 employs the steerable device 102 with the steerable tip 124 inside a passageway or anatomical lumen (e.g., sinus passage). The device 102 further includes an insertion stage 128 that translates the device 102 along a main axis inside the body. In one embodiment of the steerable tip 124, the device 102 can be configured to implement steering in one plane using one joint. In another embodiment of the steerable tip 124, the device 102 can be configured to implement yaw and pitch motion using two joints. In another embodiment, two or more parallel motors may be employed to implement the steering angle. In still another embodiment, a tendon driven system with two or more tendons embedded in the device 102 and coupled to actuators/motors at a distal end of the tendons can provide steering. In yet another embodiment, additional rotational degrees of freedom can rotate the device 102 around a primary axis (longitudinal axis) of the device. One or more of these actuation and/or rotation schemes may be combined with any one or more other actuation and/or rotation schemes, as needed.

The device control system 130 may be stored in memory 116 and be configured to translate the angle of joints 108 into actuator commands of the device or generate actuator commands to change the angle of the joints in accordance with image feedback. The device control system 130 includes a kinematic model 132 of the device and control schemes that are known in art. The kinematic model 132 computes a configuration needed for guiding the device 102 through a passageway. Parameters such as speed, position, and other spatial considerations (e.g., angles due to internal volume structures) are considered by the model 132. For example, the device control system 130 controls an amount of rotation of the joint 108 based upon a position and speed of the medical device 102 as the device approaches a branching structure, bifurcation, etc. When the next configuration is determined, actuator commands are generated by the device control system 130 to steer the device 102 by adjusting the steerable tip 124. Navigation of the device 102 in accordance with the present principles can proceed at an increased rate, which results in reduced operation times.

Workstation 112 includes a display 118 for viewing the internal images 144 and 142 of the subject (patient) or volume 134 and may include the images 142, 144 with overlays or other renderings. Display 118 may also permit a user to interact with the workstation 112 and its components and functions, or any other element within the system 100. This is further facilitated by a user interface 120 which may include a keyboard, mouse, a joystick, a haptic device, or any other peripheral or control to permit user feedback from and interaction with the workstation 112. In one embodiment, an imaging system 110 may be present for obtaining preoperative images 142 (e.g., MRI, CT, etc.). In other embodiments, the imaging system 110 may be located separately, and images may be collected remotely from other described operations. The intra-operative imaging system 111 may include a fiber optic scope, a camera system, an X-ray imaging system, a mobile X-ray imaging system, etc. for obtaining intraoperative images 144.

The device control system 130 translates the angle of joint(s) 108 into actuator commands for the device 102 using the kinematic model 132 to select pathways and steer the device 102. In one method, the images (142, 144) differentiate between open pathways and tissues. The device control system 130 selects open pathways that lead to a target location using both preoperative images 142 and intraoperative images 144. In one embodiment, the intraoperative imaging system 111 may include a mobile X-ray system for imaging of the anatomy and the device 102, a fiber optic endoscope inserted through the device lumen or integrated into the device, or other imaging configurations and technologies.

The image control system 106 is configured to integrate preoperative 3D images 142 (CT, MRI, etc.) and intraoperative images 144 (X-ray, endoscope, etc.) and register those into a single coordinate system of the robot device 102. The image control system 106 is further configured to permit the user to plan a path to an affected sinus or other target or to identify a target. In one embodiment, a path is planned and locations and angles identified for tip steering based on position within the anatomy. During the planning stage, an instruction set of commands for steering control can be generated. During operation, these commands are communicated to the device control system 130. The commands are associated with position in the anatomy or other signposts to enable the issuance of a command at the correct time to select a pathway using the commands to control the steerable tip.

Steering may be in accordance with a plan 150 stored in memory 116. The plan 150 may be selected in virtual space (e.g., using preoperative images 142). The steering control may be performed in real-time using the device control system 130 to make path determinations and angle adjustment as the device 102 is advanced.

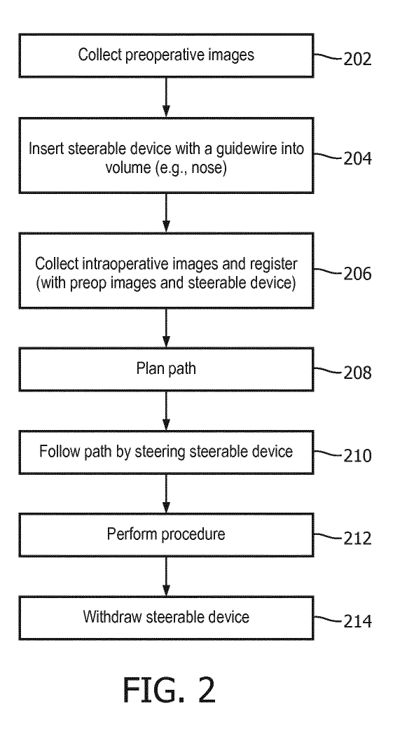

Referring to FIG. 2, a method for steering a robot is provided in accordance with illustrative embodiments. This method may be executed using the system 100 of FIG. 1. In block 202, a preoperative 3D image is taken and an affected sinus or other target is identified. In block 204, a steerable device (e.g., robot) with a guidewire is placed in the steerable device lumen and is inserted in the anatomy (e.g., the nose). This may be performed manually.

In block 206, position or image feedback is collected for the steerable device within the volume. For example, an X-ray image of the steerable device is acquired and registration is performed (e.g., registration of preoperative images to intraoperative images and the steerable device). The registration between the steerable device and X-ray system can be performed using methods known in art. In another embodiment, in block 206, an endoscope image is acquired and registration is performed. The registration between the device and endoscope images can be performed using methods known in art. In another embodiment, a position of the steerable device may be determined (e.g., using fiber optic positioning, electromagnetic positioning, image positioning, etc.). The position of the steerable device may be employed for navigating the steerable device in the volume (with or without images).

In block 208, a user/surgeon identifies a location of the affected sinus or target in one of the images (e.g., CT). Path planning is performed to determine an interactive path. The path planning may include using the image control system to compute all possible paths from the nose (or other orifice) to the sinus (or other target). In block 210, the user/surgeon follows the planned path in the volume (e.g., nasal cavity) by steering and employing a translation stage of the device (102) to advance the device tip. The translation stage can be manual (handheld, sliding stage, etc.) or motorized (with a motion trigger or speed regulation). The steerable device is automatically navigated and the steering is controlled by the control system in accordance with a plan or in real-time using position or image feedback. In one embodiment, the image control system receives the device position from the device control system and computes the tip position in the coordinate system of the path. With each computation cycle, the device control system computes whether the steerable tip needs to be actuated. If the tip position is not actuated, the device will continue to proceed along the previous path direction. If the device control system determines a change in direction is needed, the angle and direction for a given position is changed to steer the steerable tip. The device control system automatically steers the tip to comply with the desired or planned path.

In block 212, depending on the procedure, treatment or other activities are conducted on the target area. In one embodiment, once the target is achieved, the steerable device is withdrawn and a balloon is guided using a guidewire placed through the steerable device. With the balloon placed, the balloon may be expanded to open up the sinus or other anatomical feature. In block 214, upon completion of the procedure, the device is withdrawn. The device withdrawal may also employ the steering capability of the device. While described in terms a nasal procedure, it should be understood that the present principles are applicable to any procedure and are especially useful for any navigation in constrained spaces.

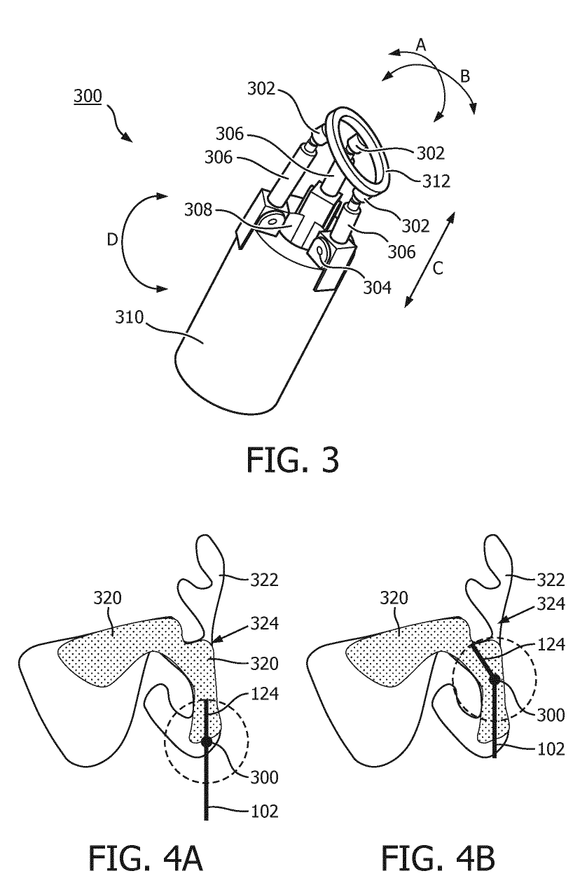

Referring to FIG. 3, a robotic feature 300 is illustratively shown in accordance with one example. The feature 300 is included in the device 102 and provides translation and rotation motions for a tip of the device 102. The feature 300 includes a shaft 310, which may include an internal lumen 308 to receive a guidewire (or catheter) or other elongated instruments. The feature 300 is employed to steer a distal end portion of the steerable device (102). In other embodiments, the feature 300 is covered by a sheath or the like. In one particularly useful embodiment, the feature 300 is part of a catheter and receives a guidewire within the internal lumen. Once the guidewire and the steerable device are in place, the steerable device (and feature 300) is/are withdrawn. The guidewire is then employed to guide a balloon catheter to the target location where the balloon is employed to expand the cavity for treatment.

The feature 300 includes an end effector 312 that may include a ring or other shape that encircles a catheter or other device passing through the internal lumen 308. The end effector 312 may be employed to direct the catheter or other instrument passing through the internal lumen 308.

The end effector 312 is coupled to translatable rods 306 (tendons) by joints 302. The translatable rods 306 can advance or retract into the shaft 310 to provide a translation motion in the direction of arrow “C”. For example, when all three of the rods 306 are advanced (or retracted) concurrently, translation is realized. If the rods 306 are advanced or retracted at different rates or for different amounts, the relative motion will provide a rotation of the end effector 312 in the direction or directions of arrows “A” and/or “B”. In addition, a rotary platform 304 may be employed to cause the entire end effector 312 to rotate about a longitudinal axis of the shaft 310 (e.g., in the direction of arrow “D”). The feature 300 provides a plurality of degrees of freedom at a localized position. In this way, accurate and well-controlled steering of the device 102 can be achieved.

While FIG. 3 shows an illustrative joint, it should be understood that more complex or simpler joints may be employed. These other joint types may include simple hinge joints, rotary joints, translational mechanisms, etc.

Referring to FIG. 4A, an illustrative example of a steerable device 102 is shown in a first configuration. The first configuration shows the steerable device 102 after insertion in a nasal cavity 320. As the device 102 approaches a bifurcation or pathway split 324, the device control system automatically senses that a steering action is needed to steer the tip 124 to comply with a desired or planned path, or the device control system senses that a particular pathway needs to be navigated in accordance with the plan. The device control mechanism employs signal control to adjust the feature 300 to provide appropriate navigation of the device 102 by controlling the angles of the tip 124.

Referring to FIG. 4B, the steerable device 102 is shown in a second configuration. The second configuration shows the steerable device 102 after a command is issued by the device control system to rotate the tip 124 using the feature 300 to control the insertion in a particular direction in the nasal cavity 320. As the device 102 approaches the bifurcation or pathway split 324, the device control system automatically steers the tip 124 to comply with the planned path or senses that pathway is the better path to achieve the present goal or target.

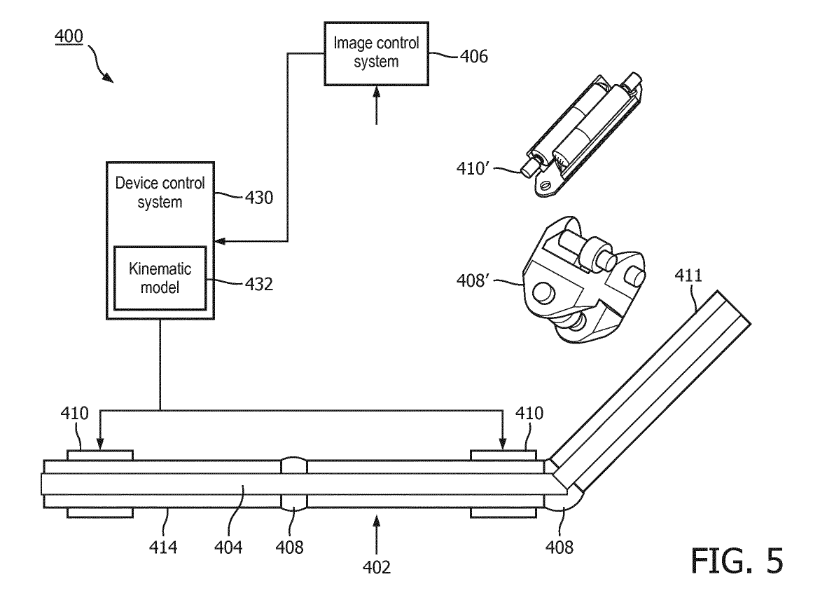

Referring to FIG. 5, a robot 400 is shown in accordance with the present principles. The robot 400 includes a steerable device 402 (see also, device 102) having one or more robotically controlled joints 408 configured to steer the device 402. The device 402 includes a lumen 404 for storing other instruments, such as a guidewire or the like. Each joint 408 may include a motor or motors 410 associated with it. The motors 410 receive signals generated in accordance with control commands to control the joints 408.

A device control system 430 (see also, system 130) is configured to receive feedback from an image control system 406 (see also, system 106) to evaluate positioning of the steerable device 402 in a volume such that control commands are issued to the one or more robotically controlled joints 408 to steer the steerable device 402 in a direction consistent with navigation of the medical device toward a target or in accordance with a plan.

The image control system 406 registers preoperative and intraoperative images to locate the position of the steerable device in a single coordinate system. The intraoperative images may include a camera image (endoscopy), an X-ray image or other imaging modality images.

The device control system 430 controls translation and/or rotation of the one or more robotically controlled joints 408 to bias the medical device toward a pathway. The device control system 430 can also control an amount of translation and/or rotation based upon a position, direction and speed of the steerable device 402 as the steerable device 402 approaches a branching structure. The device control system 430 includes a kinematic model 432 to evaluate dynamics of the steerable device 402 to control the one or more robotically controlled joints 408. The kinematic model 432 is employed to anticipate a next turn or configuration to be taken by the steerable device 402.

The one or more robotically controlled joints 408 may include one, two or more degrees of rotation. The steerable device 402 may also include a translation stage 414 to support advancing and/or retracting of the steerable device 402. The one or more robotically controlled joints 408 may include a steerable tip or end effector 411 or other distally mounted structure on the robot 400. The end effector 411 may include a plurality of translatable rods such that positions of the rods provide a rotation of the end effector 411 relative to a longitudinal axis of a shaft that supports the rods (FIG. 3).

In one embodiment, the steerable tip 411 can be configured to implement yaw and pitch motion using two motors 410′ (for one or more motors 410) and a universal joint 408′ (for one or more joints 408). Two or more parallel motors 410′may be employed to implement the steering angle. In another embodiment, a tendon driven system (300) with two or more tendons embedded in the device 102 and coupled to actuators/motors at a distal end of the tendons can provide steering. In yet another embodiment, additional rotational degrees of freedom can rotate the device 402 around a primary axis (longitudinal axis) of the device 402. One or more of these actuation and/or rotation schemes may be combined with any one or more other actuation and/or rotation schemes, as needed.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for an image guided robotic guide catheter placement (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims.

Having thus described the details and particularity required by the patent laws, what is claimed and desired protected by Letters Patent is set forth in the appended claims:

1. A robot, comprising:
a steerable device having one or more robotically controlled joints configured to steer the steerable device; and
a device control system configured to adjust positioning of the steerable device in accordance with one of image feedback from an image control system or a plan such that control commands are issued to the one or more robotically controlled joints to steer the steerable device in a direction consistent with navigation of the steerable device toward a target.

2. The robot as recited in claim 1, wherein the one or more robotically controlled joints includes at least two degrees of rotation.

3. The robot as recited in claim 1, wherein the image control system registers preoperative and intraoperative images to locate a position of the steerable device in a single coordinate system.

4. The robot as recited in claim 3, wherein the intraoperative images include one of a camera image or an X-ray image.

5. The robot as recited in claim 1, wherein the device control system controls rotation of the one or more robotically controlled joints to steer the steerable device toward a pathway.

6. The robot as recited in claim 1, wherein the device control system controls an amount of rotation of the one or more robotically controlled joints based upon a position, direction and speed of the steerable device as the steerable device approaches a branching structure.

7. The robot as recited in claim 1, wherein the one or more robotically controlled joints includes an end effector that includes a plurality of translatable rods such that positions of the rods provide a rotation of the end effector relative to a longitudinal axis of a shaft that supports the rods.

8. The robot as recited in claim 1, wherein the device control system includes a kinematic model to evaluate dynamics to control the one or more robotically controlled joints.

9. A guidance system, comprising:
a steerable device having an adjustable tip portion, the tip portion being coupled to a robotically controlled joint;
an image control system configured to combine intraoperative images with preoperative images to evaluate a position of the steerable device within a volume; and
a device control system configured to receive position information from the image control system and to evaluate positioning of the steerable device using a kinematic model, the device control system issuing control commands to the robotically controlled joint to steer the steerable device in a direction consistent with navigation of the steerable device toward a target.

10. (canceled)

11. The guidance system as recited in claim 9, wherein the image control system registers the preoperative and the intraoperative images to locate a position of the steerable device in a single coordinate system.

12. The guidance system as recited in claim 9, wherein the device control system controls rotation of the robotically controlled joint to steer the steerable device toward a pathway.

13. The guidance system as recited in claim 9, wherein the device control system controls an amount of rotation based upon a position, direction and speed of the medical device as the device approaches a branching structure.

14. (canceled)

15. The guidance system as recited in claim 9, wherein the robotically controlled joint includes an end effector that includes a plurality of translatable rods such that positions of the rods provide a rotation of the end effector relative to a longitudinal axis of a shaft that supports the rods.

16. A guidance method, comprising:
inserting a steerable device having an adjustable robotically controlled joint configured to be steered into a volume;
providing position or image feedback of the steerable device within the volume; and
automatically navigating the steerable device toward a target in accordance with a plan using a device control system configured to receive the feedback, to evaluate positioning of the steerable device in the volume and to issue control commands to the robotically controlled joint to steer the steerable device.

17. The guidance method as recited in claim 16, wherein the robotically controlled joint includes at least two degrees of rotation.

18. The guidance method as recited in claim 16, further comprising registering preoperative and intraoperative images to locate a position of the steerable device in a single coordinate system.

19. The guidance method as recited in claim 16, wherein the device control system controls an amount of rotation of the steerable device based upon a position, direction and speed of the steerable device as the steerable device approaches a branching structure.

20. The guidance method as recited in claim 16, wherein the robotically controlled joint includes an end effector that includes a plurality of translatable rods such that positions of the rods provide a rotation of the end effector relative to a longitudinal axis of a shaft that supports the rods.

21. The guidance method as recited in claim 16, wherein the device control system includes a kinematic model to evaluate dynamics to control the robotically controlled joint.

22. The guidance method as recited in claim 16, wherein the inserting is performed manually by an operator, and the automatically navigating includes steering controlled by the control system in accordance with a plan.
